(19) United States
(12) Patent Application Publication
(10) Pub. No.: US 2018/0263527 A1
(43) Pub. Date: Sep. 20, 2018

(54) ENDOSCOPE POSITION SPECIFYING DEVICE, METHOD, AND PROGRAM

(71) Applicant: FUJIFILM Corporation, Tokyo (JP)

(72) Inventor: Yoshiro KITAMURA, Tokyo (JP)

(73) Assignee: FUJIFILM Corporation, Tokyo (JP)

(21) Appl. No.: 15/868,045

(22) Filed: Jan. 11, 2018

(30) Foreign Application Priority Data
Mar. 16, 2017 (JP) 2017-051506

(51) Int. Cl. A61B 5/06; A61B 1/00; A61B 1/04; A61B 1/267

(52) U.S. Cl. CPC A61B 5/066; A61B 1/00009; A61B 1/2676; A61B 1/00006; A61B 1/04; A61B 1/0005

(57) ABSTRACT

An image acquisition unit sequentially acquires an endoscope image of a tubular structure having a plurality of branch structures, and an image generation unit generates an image of the tubular structure. A first certainty factor calculation unit calculates a first certainty factor indicating a possibility of presence of the endoscope within the tubular structure. A second certainty factor calculation unit calculates a second certainty factor indicating a possibility of presence of the endoscope by performing matching between the image of the tubular structure and each of the endoscope images at each of a plurality of positions within the tubular structure. A current position specifying unit specifies the current position of the endoscope based on the first and second certainty factors.

### CROSS REFERENCE TO RELATED APPLICATIONS

The present application claims priority under 35 U.S.C. § 119 to Japanese Patent Application No. 2017-051506 filed on Mar. 16, 2017. The above application is hereby expressly incorporated by reference, in its entirety, into the present application.

### BACKGROUND

### Field of the Invention

The present invention relates to an endoscope position specifying device, method, and program for specifying the position of an endoscope in a tubular structure having branch structures, such as a bronchus, in the case of observing the tubular structure by inserting the endoscope into the tubular structure.

### Description of the Related Art

In recent years, a technique of observing or treating a tubular structure, such as a bronchus and a large intestine of a patient, using an endoscope has been drawing attention. However, in the endoscope image, an image in which the color or texture of the inside of the tubular structure is clearly expressed by an imaging element, such as a charge coupled device (CCD), can be obtained, while the inside of the tubular structure is expressed as a two-dimensional image. For this reason, it is difficult to ascertain which position in the tubular structure the endoscope image represents. In particular, since a bronchial endoscope has a small diameter and accordingly has a narrow field of view, it is difficult to make the distal end of the endoscope reach a target position.

Therefore, a method of navigating an endoscope using a three-dimensional image acquired by tomographic imaging using a modality, such as a computed tomography (CT) apparatus or a magnetic resonance imaging (Mill) apparatus, has been proposed. For example, WO2012-101888A has proposed a method of generating a virtual endoscope image matching the real endoscope image of the bronchus, calculating the direction, angle, and the like of the endoscope distal end based on a parameter at the time of generating the virtual endoscope image, and detecting the position of the endoscope distal end on the graph structure of the bronchus. JP2016-179121A has proposed a method of detecting the passing position of the endoscope by extracting the graph structure of the bronchus from a three-dimensional image and performing matching between the real endoscope image at the branching position of the bronchus and the three-dimensional image in the bronchus. JP2014-000421A has proposed a method in which the amount of movement of an endoscope is calculated based on the position of a characteristic structure characterizing a local part on the luminal mucosa included in the real endoscope image of preceding and subsequent imaging times, for example, the position of luminal mucosa wrinkles and blood vessels seen through the surface.

### SUMMARY

Branch structures included in the bronchus have similar shapes regardless of their positions. Therefore, in a case where the matching between the real endoscope image and the three-dimensional image is performed as in the methods disclosed in WO2012-101888A and JP2016-179121A, a plurality of virtual endoscope images similar to branch structures included in the real endoscope image may be detected. In such a case, the position of the endoscope differs greatly depending on which of the virtual endoscope images is used for navigation. In addition, although the current position of the endoscope can be detected by the method disclosed in JP2014-000421A, an error is accumulated as the time passes. As a result, the detected position of the endoscope may gradually deviate from the actual position.

The invention has been made in view of the above circumstances, and it is an object of the invention to more accurately specify the position of an endoscope inserted into a tubular structure having branch structures.

An endoscope position specifying device according to the invention comprises: endoscope image acquisition unit for sequentially acquiring endoscope images that are generated by an endoscope inserted into a tubular structure having a plurality of branch structures and that show an inner wall of the tubular structure; image generation unit for generating an image of the tubular structure from a three-dimensional image including the tubular structure; first certainty factor calculation unit for calculating an amount of movement of the endoscope during a period from acquisition of a reference endoscope image to acquisition of a latest endoscope image based on the sequentially acquired endoscope images, estimating a position of the endoscope based on the calculated amount of movement, and calculating a first certainty factor indicating a possibility of presence of the endoscope within the tubular structure based on the estimated position; second certainty factor calculation unit for calculating a second certainty factor, which indicates a possibility of presence of the endoscope, at each of a plurality of positions within the tubular structure by performing matching between the image of the tubular structure and each of the endoscope images at each of the plurality of positions within the tubular structure; and current position specifying unit for specifying a current position of the endoscope based on the first and second certainty factors.

In the endoscope position specifying device according to the invention, the second certainty factor calculation unit may calculate the second certainty factor in a predetermined range with the position of the endoscope estimated by the first certainty factor calculation unit as a reference.

The endoscope position specifying device according to the invention may further comprise normal endoscope image specifying unit for specifying normal endoscope images among the sequentially acquired endoscope images. The first certainty factor calculation unit may calculate the first certainty factor by selecting the reference endoscope image and the latest endoscope image from the normal endoscope images.

Usually, an endoscope image captured by an endoscope apparatus shows the structure of the inner wall of a tubular structure. However, in an endoscopic examination, liquid such as drug or water may be ejected from the distal end of the endoscope. In such a case, the endoscope image includes droplets of the ejected liquid, but does not include the inner wall of the tubular structure. Accordingly, the endoscope image is an image that is meaningless in diagnosis. An endoscope image that does not include the inner wall of the tubular structure, which is important for diagnosis and which should be originally included, is referred to as an “abnormal endoscope image”.

A “normal endoscope image” means an endoscope image that includes the inner wall of the tubular structure, which is important for diagnosis and which should be originally included.

In the endoscope position specifying device according to the invention, the first certainty factor calculation unit may set a plurality of the reference endoscope images, calculate a plurality of amounts of movement of the endoscope during a period from acquisition of each of the plurality of reference endoscope images to acquisition of the latest endoscope image, estimate a plurality of positions of the endoscope from the plurality of amounts of movement, and calculate the first certainty factor at each of the plurality of estimated positions. The current position specifying unit may specify the current position of the endoscope based on a plurality of the first certainty factors and the second certainty factor.

The endoscope position specifying device according to the invention may further comprise display control unit for displaying the image of the tubular structure and displaying the current position of the endoscope on the image of the tubular structure.

An endoscope position specifying method according to the invention comprises: sequentially acquiring endoscope images that are generated by an endoscope inserted into a tubular structure having a plurality of branch structures and that show an inner wall of the tubular structure; generating an image of the tubular structure from a three-dimensional image including the tubular structure; calculating an amount of movement of the endoscope during a period from acquisition of a reference endoscope image to acquisition of a latest endoscope image based on the sequentially acquired endoscope images, estimating a position of the endoscope based on the calculated amount of movement, and calculating a first certainty factor indicating a possibility of presence of the endoscope within the tubular structure based on the estimated position; calculating a second certainty factor, which indicates a possibility of presence of the endoscope, at each of a plurality of positions within the tubular structure by performing matching between the image of the tubular structure and each of the endoscope images at each of the plurality of positions within the tubular structure; and specifying a current position of the endoscope based on the first and second certainty factors.

In addition, a program causing a computer to execute the endoscope position specifying method according to the present invention may be provided.

Another endoscope position specifying device according to the invention comprises: a memory for storing a command to be executed by a computer; and a processor configured to execute the stored command. The processor executes: endoscope image acquisition processing for sequentially acquiring endoscope images that are generated by an endoscope inserted into a tubular structure having a plurality of branch structures and that show an inner wall of the tubular structure; image generation processing for generating an image of the tubular structure from a three-dimensional image including the tubular structure; first certainty factor calculation processing for calculating an amount of movement of the endoscope during a period from acquisition of a reference endoscope image to acquisition of a latest endoscope image based on the sequentially acquired endoscope images, estimating a position of the endoscope based on the calculated amount of movement, and calculating a first certainty factor indicating a possibility of presence of the endoscope within the tubular structure based on the estimated position; second certainty factor calculation processing for calculating a second certainty factor, which indicates a possibility of presence of the endoscope, at each of a plurality of positions within the tubular structure by performing matching between the image of the tubular structure and each of the endoscope images at each of the plurality of positions within the tubular structure; and current position specification processing for specifying a current position of the endoscope based on the first and second certainty factors.

According to the invention, the amount of movement of the endoscope during a period from the acquisition of the reference endoscope image to the acquisition of the latest endoscope image is calculated based on the sequentially acquired endoscope images, the position of the endoscope is estimated based on the calculated amount of movement, and the first certainty factor indicating the possibility of presence of the endoscope within the tubular structure is calculated based on the estimated position. Then, matching between the image of the tubular structure and the endoscope image is performed at each of a plurality of positions within the tubular structure, so that the second certainty factor indicating the possibility of presence of the endoscope is calculated at each of the plurality of positions. Using the first certainty factor, a relative change in the position of the endoscope from the acquisition position of the reference endoscope image can be accurately calculated. However, as the time passes, an error may be accumulated to lower the accuracy. On the other hand, using the second certainty factor, the absolute position of the endoscope can be accurately calculated. However, a plurality of branches having similar shapes are included in the tubular structure. For this reason, the second certainty factor becomes large at a plurality of positions within the tubular structure. As a result, there is a possibility that the current position of the endoscope cannot be specified.

In the present embodiment, since the current position of the endoscope is specified based on both the first and second certainty factors, it is possible to more accurately specify the position of the endoscope inserted into the tubular structure having branch structures by taking advantage of the first and second certainty factors.

### BRIEF DESCRIPTION OF THE DRAWINGS

FIG. 1 is a hardware configuration diagram showing the outline of a diagnostic assistance system to which an endoscope position specifying device according to a first embodiment of the invention is applied.

FIG. 2 is a diagram showing the schematic configuration of the endoscope position specifying device according to the first embodiment realized by installing an endoscope position specifying program on a computer.

FIG. 3 is a schematic block diagram showing the configuration of a first certainty factor calculation unit.

FIG. 4 is a diagram showing an endoscope image.

FIG. 5 is a diagram illustrating the calculation of the deviation of an endoscope distal end.

FIG. 6 is a diagram illustrating the estimation of the position of an endoscope distal end.

FIG. 7 is a diagram showing the distribution of a first certainty factor.

FIG. 8 is a diagram showing the distribution of the first certainty factor in a bronchus image.

FIG. 9 is a diagram showing a range for generating a virtual branch image.

FIG. 10 is a diagram showing a virtual branch image.

FIG. 11 is a diagram illustrating the calculation of a second certainty factor.

FIG. 12 is a diagram showing an image displayed on a display.

FIG. 13 is a flowchart showing the process performed in the first embodiment.

FIG. 14 is a diagram showing the position of an endoscope estimated based on a plurality of reference endoscope images in a second embodiment.

FIG. 15 is a diagram showing an abnormal endoscope image.

FIG. 16 is a diagram showing the schematic configuration of an endoscope position specifying device according to a third embodiment.

FIG. 17 is a diagram illustrating the specification of a normal endoscope image.

### DESCRIPTION OF THE PREFERRED EMBODIMENTS

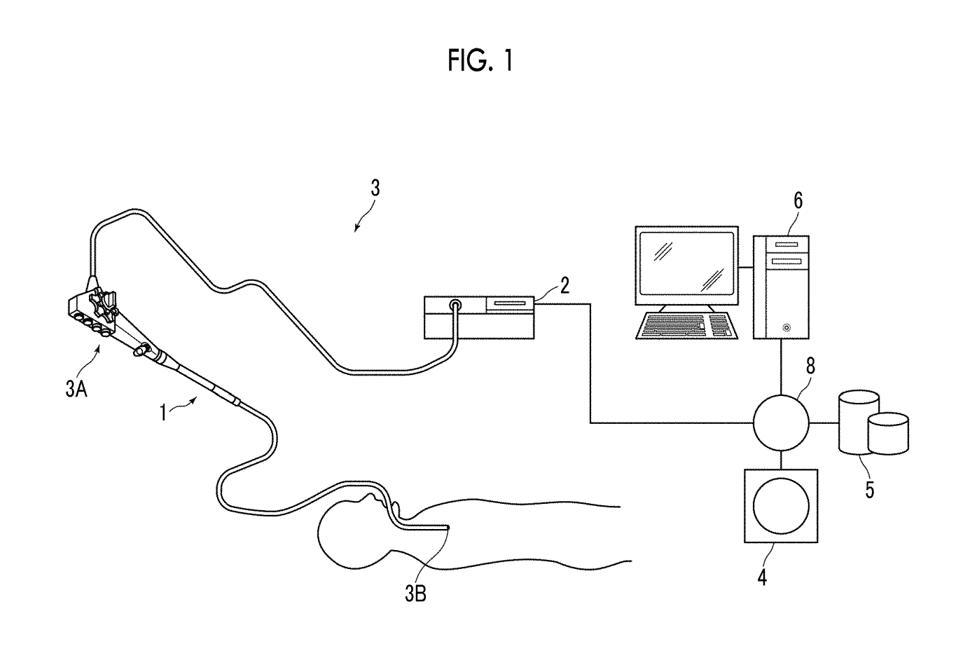

Hereinafter, embodiments of the invention will be described with reference to the accompanying diagrams. FIG. 1 is a hardware configuration diagram showing the outline of a diagnostic assistance system to which an endoscope position specifying device according to a first embodiment of the invention is applied. As shown in FIG. 1, in this system, an endoscope apparatus 3, a three-dimensional image capturing apparatus 4, an image storage server 5, and an endoscope position specifying device 6 are connected to each other in a communicable state through a network 8.

The endoscope apparatus 3 includes an endoscope scope 1 for imaging the inside of a tubular structure of a subject, a processor device 2 for generating an image of the inside of the tubular structure based on a signal obtained by imaging, and the like.

The endoscope scope 1 is obtained by continuously attaching an insertion part, which is inserted into the tubular structure of the subject, to an operation unit 3A, and is connected to the processor device 2 through a universal cord detachably connected to the processor device 2. The operation unit 3A includes various buttons for giving an instruction for an operation to make a distal end 3B of the insertion part curve in a vertical direction and a horizontal direction within a predetermined angular range, or for collecting samples of tissues by operating an insertion needle attached to the distal end of the endoscope scope 1, or for spraying a medicine. In the present embodiment, the endoscope scope 1 is a flexible mirror for bronchi, and is inserted into the bronchus of the subject. Then, light guided through an optical fiber from a light source device (not shown) provided in the processor device 2 is emitted from the distal end 3B of the insertion part of the endoscope scope 1, and an image of the inside of the bronchus of the subject is acquired by the imaging optical system of the endoscope scope 1. In order to facilitate the explanation, the distal end 3B of the insertion part of the endoscope scope 1 will be referred to as an endoscope distal end 3B in the following explanation.

The processor device 2 generates an endoscope image G0 by converting an imaging signal captured by the endoscope scope 1 into a digital image signal and correcting the image quality by digital signal processing, such as white balance adjustment and shading correction. The generated image is a moving image configured to include a plurality of endoscope images G0 expressed at a predetermined frame rate, such as 30 fps. The endoscope image G0 is transmitted to the image storage server 5 or the endoscope position specifying device 6.

The three-dimensional image capturing apparatus 4 is an apparatus that generates a three-dimensional image V0 showing a part, which is an examination target part of a subject, by imaging the part. Specifically, the three-dimensional image capturing apparatus 4 is a CT apparatus, an MRI apparatus, a positron emission tomography (PET) apparatus, an ultrasound diagnostic apparatus, or the like. The three-dimensional image V0 generated by the three-dimensional image capturing apparatus 4 is transmitted to the image storage server 5 and is stored therein. In the present embodiment, the three-dimensional image capturing apparatus 4 is a CT apparatus that generates the three-dimensional image V0 by imaging the chest including a bronchus.

The image storage server 5 is a computer that stores and manages various kinds of data, and includes a large-capacity external storage device and software for database management. The image storage server 5 transmits and receives image data and the like by performing communication with other apparatuses through the network 8. Specifically, the image storage server 5 acquires image data, such as the endoscope image G0 acquired by the endoscope apparatus 3 and the three-dimensional image V0 generated by the three-dimensional image capturing apparatus 4, through the network, and stores the image data in a recording medium, such as a large-capacity external storage device and manages the image data. The endoscope image G0 is moving image data sequentially acquired according to the movement of the endoscope distal end 3B. Therefore, it is preferable that the endoscope image G0 is transmitted to the endoscope position specifying device 6 without passing through the image storage server 5. The storage format of image data or the communication between apparatuses through the network 8 is based on protocols, such as a digital imaging and communication in medicine (DICOM).

The endoscope position specifying device 6 is realized by installing an endoscope position specifying program of the first embodiment on one computer. The computer may be a workstation or a personal computer that is directly operated by a doctor who performs diagnosis, or may be a server computer connected to these through a network. The endoscope position specifying program is distributed by being recorded on a recording medium, such as a digital versatile disc (DVD) or a compact disk read only memory (CD-ROM), and is installed onto the computer from the recording medium. Alternatively, the endoscope position specifying program is stored in a storage device of a server computer connected to the network or in a network storage so as to be accessible from the outside, and is downloaded and installed onto a computer used by a doctor, who is a user of the endoscope position specifying device 6, when necessary.

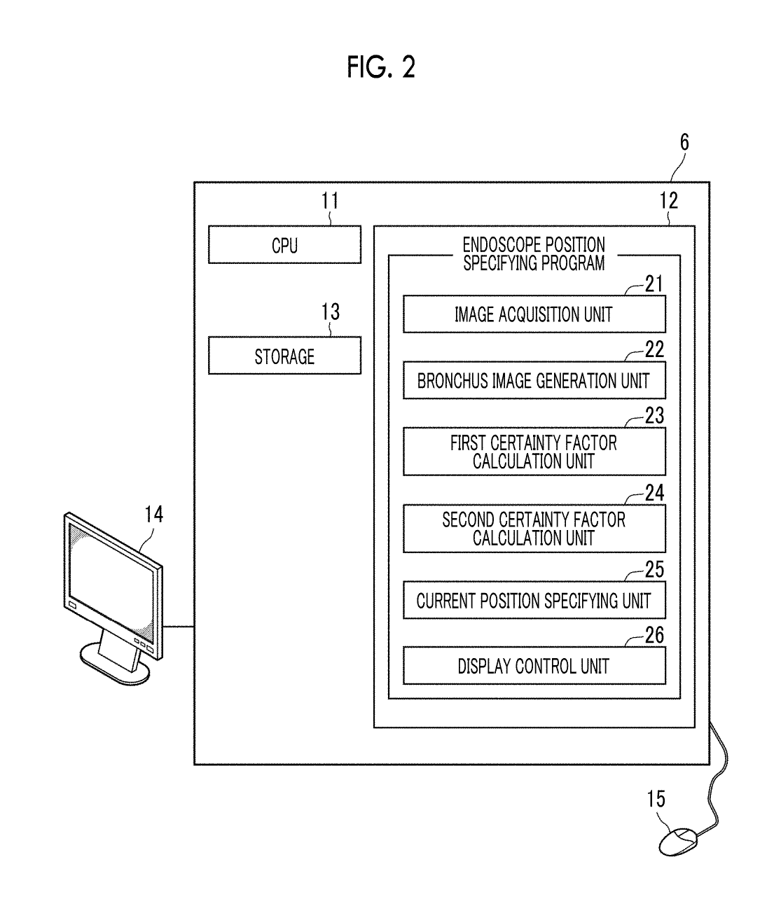

FIG. 2 is a diagram showing the schematic configuration of an endoscope position specifying device realized by installing an endoscope position specifying program on a computer. As shown in FIG. 2, the endoscope position specifying device 6 includes a central processing unit (CPU) 11, a memory 12, and a storage 13 as the configuration of a standard workstation. A display 14 and an input unit 15, such as a mouse, are connected to the endoscope position specifying device 6.

The endoscope image G0 and the three-dimensional image V0, which are acquired from the endoscope apparatus 3, the three-dimensional image capturing apparatus 4, the image storage server 5, and the like through the network 8, and the image generated by the processing in the endoscope position specifying device 6, and the like are stored in the storage 13.

The endoscope position specifying program is stored in the memory 12. As processing to be executed by the CPU 11, the endoscope position specifying program defines: image acquisition processing for sequentially acquiring the endoscope image G0 generated by the processor device 2 and acquiring image data, such as the three-dimensional image V0 generated by the three-dimensional image capturing apparatus 4; bronchus image generation processing for generating a bronchus image, which is an image of a tubular structure, from the three-dimensional image V0; first certainty factor calculation processing for calculating the amount of movement of the endoscope during a period from the acquisition of a reference endoscope image to the acquisition of the latest endoscope image based on the sequentially acquired endoscope images, estimating the position of the endoscope based on the calculated amount of movement, and calculating a first certainty factor indicating the possibility of presence of the endoscope within the tubular structure based on the estimated position; second certainty factor calculation processing for calculating a second certainty factor indicating the possibility of presence of the endoscope at each of a plurality of positions within the tubular structure by performing matching between the image of the tubular structure and the endoscope image at each of the plurality of positions within the tubular structure; current position specification processing for specifying the current position of the endoscope based on the first and second certainty factors; and display control processing for displaying the bronchus image and displaying the current position of the endoscope on the bronchus image.

The CPU 11 executes these processes according to the program, so that the computer functions as an image acquisition unit 21, a bronchus image generation unit 22, a first certainty factor calculation unit 23, a second certainty factor calculation unit 24, a current position specifying unit 25, and a display control unit 26. The endoscope position specifying device 6 may include a plurality of processors that perform image acquisition processing, bronchus image generation processing, first certainty factor calculation processing, second certainty factor calculation processing, current position specification processing, and display control processing. Here, the image acquisition unit 21 corresponds to endoscope image acquisition unit, and the bronchus image generation unit 22 corresponds to an image generation unit.

The image acquisition unit 21 sequentially acquires the endoscope image G0 by imaging the inside of the bronchus using the endoscope apparatus 3, and acquires the three-dimensional image V0. In a case where the three-dimensional image V0 is already stored in the storage 13, the image acquisition unit 21 may acquire the three-dimensional image V0 from the storage 13. The endoscope image G0 is displayed on the display 14. The image acquisition unit 21 stores the acquired endoscope image G0 and the acquired three-dimensional image V0 in the storage 13.

The bronchus image generation unit 22 generates a bronchus image from the three-dimensional image V0. Therefore, the bronchus image generation unit 22 generates a three-dimensional bronchus image by extracting a graph structure of a bronchial region included in the three-dimensional image V0 using the method disclosed in JP2010-220742A or the like, for example. Hereinafter, an example of the graph structure extraction method will be described.

In the three-dimensional image V0, pixels inside the bronchus are expressed as a region showing low pixel values since the pixels correspond to an air region. However, the bronchial wall is expressed as a cylindrical or linear structure showing relatively high pixel values. Therefore, the bronchus is extracted by performing structural analysis of the shape based on the distribution of pixel values for each pixel.

The bronchus branches in multiple stages, and the diameter of the bronchus decreases as the distance from the distal end decreases. The bronchus image generation unit 22 generates a plurality of three-dimensional images with different resolutions by performing multi-resolution conversion of the three-dimensional image V0 so that bronchi having different sizes can be detected, and applies a detection algorithm for each three-dimensional image of each resolution, thereby detecting tubular structures having different sizes.

First, at each resolution, a Hessian matrix of each pixel of the three-dimensional image is calculated, and it is determined whether or not the pixel is a pixel in the tubular structure from the magnitude relationship of eigenvalues of the Hessian matrix. The Hessian matrix is a matrix having, as its elements, partial differential coefficients of the second order of density values in directions of the respective axes (x, y, and z axes of the three-dimensional image), and is a 3×3 matrix as in the following Equation (1).

$${∇}^{2} I = [ \begin{matrix}{I}_{xx} & {I}_{xy} & {I}_{xz} \\ {I}_{xx} & {I}_{xy} & {I}_{xz} \\ {I}_{xx} & {I}_{xy} & {I}_{xz}\end{matrix} ] \\ {I}_{xx} = \frac{{δ}^{2} I}{δ {x}^{2}} , {I}_{xy} = \frac{{δ}^{2} I}{δ x δ {y}^{2}} , … \qquad ( 1 )$$

Assuming that the eigenvalues of the Hessian matrix at an arbitrary pixel are λ1, λ2, and λ3, it is known that the pixel is a tubular structure in a case where two of the eigenvalues are large and one eigenvalue is close to 0, for example, in a case where λ3, λ2>>λ1, and λ1≈0 are satisfied. In addition, an eigenvector corresponding to the minimum eigenvalue (λ1≈0) of the Hessian matrix matches a main axis direction of the tubular structure.

The bronchus can be expressed in a graph structure, but the tubular structure extracted in this manner is not necessarily detected as one graph structure, in which all tubular structures are connected to each other, due to the influence of a tumor or the like. Therefore, after the detection of the tubular structure from the three-dimensional image V0 is ended, by performing evaluation regarding whether each extracted tubular structure is within a predetermined distance and an angle between the direction of the basic line connecting arbitrary points on the two extracted tubular structures to each other and the main axis direction of each tubular structure is within a predetermined angle, it is determined whether or not a plurality of tubular structures are connected to each other, thereby reconstructing the connection relationship of the extracted tubular structures. By this reconstruction, the extraction of the graph structure of the bronchus is completed.

Then, the bronchus image generation unit 22 generates a three-dimensional graph structure showing the bronchi as a bronchus image B0 by classifying the extracted graph structure into a start point, an end point, a branch point, and a side and connecting the start point, the end point, and the branch point to each other with the side. The method of generating the bronchus image B0 is not limited to the method described above, and other methods may be adopted.

The bronchus image generation unit 22 detects the central axis of the graph structure of the bronchus. The distance from each pixel position on the central axis of the graph structure of the bronchus to the inner wall of the graph structure of the bronchus is calculated as the radius of the bronchus at the pixel position. The direction in which the central axis of the graph structure extends is a direction in which the bronchus extends.

The first certainty factor calculation unit 23 calculates the amount of movement of the endoscope during a period from the acquisition of a reference endoscope image to the acquisition of the latest endoscope image based on the sequentially acquired endoscope image G0, estimates the position of the endoscope based on the calculated amount of movement, and calculates a first certainty factor A1 indicating the possibility of presence of the endoscope distal end 3B within the bronchus based on the estimated position. Hereinafter, the calculation of the first certainty factor A1 will be described.

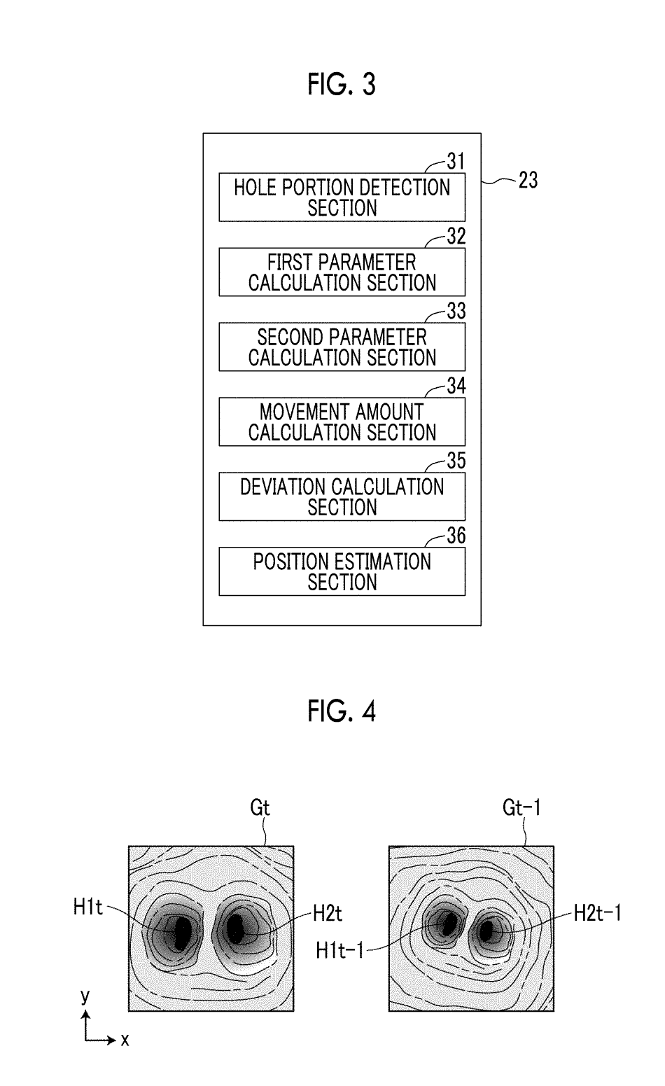

FIG. 3 is a schematic block diagram showing the configuration of the first certainty factor calculation unit. As shown in FIG. 3, the first certainty factor calculation unit 23 includes a hole portion detection section 31, a first parameter calculation section 32, a second parameter calculation section 33, a movement amount calculation section 34, a deviation calculation section 35, and a position estimation section 36.

The hole portion detection section 31 detects a hole portion of the bronchus from each of a first endoscope image and a second endoscope image, which is acquired temporally earlier than the first endoscope image, among the sequentially acquired endoscope images G0. In the following explanation, reference numerals of the first and second endoscope images are Gt and Gt-1. Therefore, the second endoscope image Gt-1 is acquired at a time immediately before the first endoscope image Gt. The second endoscope image Gt-1 is a reference endoscope image, and the first endoscope image Gt is the latest endoscope image.

FIG. 4 is a diagram showing first and second endoscope images. In a case where the first endoscope image Gt and the second endoscope image Gt-1 are compared with each other, the second endoscope image Gt-1 is acquired temporally earlier than the first endoscope image Gt. Therefore, two hole portions H1t-1 and H2t-1 at the branch of the bronchus included in the second endoscope image Gt-1 are smaller than two hole portions H1t and H2t included in the first endoscope image Gt.

The hole portion detection unit 31 detects hole portions from the first endoscope image Gt and the second endoscope image Gt-1 using the MSER method. In the MSER method, a dark region where the brightness is less than the threshold value in the endoscope image is detected. Then, a dark region where the brightness is less than the threshold value is detected while changing the threshold value. Then, in the MSER method, a threshold value at which the area of a dark region changes most largely with respect to a threshold value change is calculated, and a dark region where the brightness is less than the threshold value is detected as a hole portion.

The first parameter calculation section 32 calculates a first parameter indicating the amount of parallel movement of the first endoscope image Gt with respect to the second endoscope image Gt-1 in order to match the hole portions of the first endoscope image Gt and the second endoscope image Gt-1 with each other. Specifically, the first parameter calculation section 32 calculates a correlation while moving the first endoscope image Gt in a two-dimensional manner with respect to the second endoscope image Gt-1, with a state in which the center of gravity of the first endoscope image Gt and the center of gravity of the second endoscope image Gt-1 match each other being an initial position. Then, the two-dimensional amount of movement of the first endoscope image Gt having the maximum correlation is calculated as a first parameter P1. The first parameter P1 is x and y values in a case where the x axis is set in the horizontal direction and the y axis is set in the vertical direction on the paper surface as shown in FIG. 4.

The first parameter calculation section 32 may extract a local region including a hole portion from each of the first endoscope image Gt and the second endoscope image Gt-1, and calculate the first parameter P1 only using the extracted region. Therefore, it is possible to reduce the amount of calculation for calculating the first parameter P1. In addition, in each of the first endoscope image Gt and the second endoscope image Gt-1, the first parameter P1 may be calculated by increasing the weighting of a local region including a hole portion.

The second parameter calculation section 33 performs alignment between the first endoscope image Gt and the second endoscope image Gt-1 based on the first parameter P1, and calculates a second parameter P2 including the amount of enlargement and reduction of the first endoscope image Gt with respect to the second endoscope image Gt-1 in order to match the hole portions of the first endoscope image Gt and the second endoscope image Gt-1 after the alignment with each other. In the present embodiment, in addition to the amount of enlargement and reduction, the second parameter P2 further including the amount of rotation of the first endoscope image Gt with respect to the second endoscope image Gt-1 is calculated.

Therefore, the second parameter calculation section 33 performs alignment between the first endoscope image Gt and the second endoscope image Gt-1 based on the first parameter P1 first. Specifically, the alignment is performed by moving the first endoscope image Gt in parallel to the second endoscope image Gt-1 based on the first parameter P1.

Then, the second parameter calculation section 33 calculates a correlation while gradually enlarging and reducing the first endoscope image Gt after the alignment with respect to the second endoscope image Gt-1. In this case, in a case where the size of the hole portion included in the first endoscope image Gt matches the size of the hole portion included in the second endoscope image Gt-1, the correlation is maximized. The second parameter calculation section 33 calculates the enlargement ratio of the first endoscope image Gt having the maximum correlation as the amount of enlargement and reduction included in the second parameter P2.

The second parameter calculation section 33 calculates a correlation while gradually rotating the first endoscope image Gt after the alignment with respect to the second endoscope image Gt-1 with the center of the detected hole portion as a reference. In this case, in a case where there are a plurality of detected hole portions, the second parameter calculation section 33 calculates a correlation while gradually rotating the first endoscope image Gt after the alignment with respect to the second endoscope image Gt-1 with the center of each of the detected hole portions as a reference. The correlation may also be calculated with only the center of one detected hole portion as a reference. Then, the rotation angle of the first endoscope image Gt at the time at which the correlation is maximized is calculated as the amount of rotation included in the second parameter P2. The second parameter calculation section 33 may first calculate any of the amount of enlargement and reduction and the amount of rotation included in the second parameter P2.

Based on the first parameter P1 and the second parameter P2, the movement amount calculation section 34 calculates the amount of movement of the endoscope distal end 3B from the acquisition position of the second endoscope image Gt-1 to the acquisition position of the first endoscope image Gt. Specifically, the amount of parallel movement of the endoscope distal end 3B, the amount of movement of the endoscope distal end 3B in a direction in which the central axis of the bronchus extends, and the amount of rotational movement of the endoscope distal end 3B are calculated. Therefore, the movement amount calculation section 34 first sets the initial position of the endoscope distal end 3B in the bronchus image B0 extracted by the bronchus image generation unit 22. In the present embodiment, the initial position is the position of the first branch in the endoscope image G0 displayed on the display 14. For the setting of the initial position, the display control unit 26 displays the bronchus image B0 extracted by the bronchus image generation unit 22 extracted on the display 14. The operator sets the initial position on the bronchus image B0 displayed on the display 14 using the input unit 15. The initial position may be automatically set on the bronchus image B0 by matching the endoscope image G0 at the position of the first branch with the bronchus image.

In the present embodiment, with the initial position as a start position, the amount of movement is calculated every time the endoscope image G0 is acquired. Here, the calculation of the amount of movement using the first endoscope image Gt and the second endoscope image Gt-1 at a certain point in time will be described. The movement amount calculation section 34 calculates the amount of movement by converting the first parameter P1 and the second parameter P2 into the amount of movement of the endoscope distal end 3B. Here, the acquisition position of the second endoscope image Gt-1 is specified by the immediately preceding process in which the second endoscope image Gt-1 is the first endoscope image Gt. The movement amount calculation section 34 acquires the radius of the bronchus at the acquisition position of the second endoscope image Gt-1 from the bronchus image B0. Then, the movement amount calculation section 34 calculates the amount of parallel movement of the endoscope distal end 3B by multiplying the first parameter P1, which is the amount of parallel movement, by the acquired radius of the bronchus as a scaling coefficient. In addition, by multiplying the amount of enlargement and reduction included in the second parameter P2 by the scaling coefficient, the amount of movement of the endoscope distal end 3B in a direction in which the central axis of the bronchus extends is calculated. In a case where the amount of enlargement and reduction is an enlargement value (that is, in a case where the enlargement ratio is larger than 1), the direction of movement along the central axis of the bronchus is a direction in which the endoscope distal end 3B faces. In a case where the amount of enlargement and reduction is a reduction value (that is, in a case where the enlargement ratio is smaller than 1), the direction of movement along the central axis of the bronchus is a direction opposite to the direction in which the endoscope distal end 3B faces. For the amount of rotation included in the second parameter P2, the amount of rotation is calculated as the amount of rotational movement as it is without being multiplied by the scaling coefficient.

The movement amount calculation section 34 stores the amount of movement, that is, the amount of parallel movement of the endoscope distal end 3B, the amount of movement of the endoscope distal end 3B in a direction in which the central axis of the bronchus extends, and the amount of rotational movement of the endoscope distal end 3B, in the storage 13. In the present embodiment, the amount of movement is accumulated and stored every time the endoscope image G0 is acquired from the initial position.

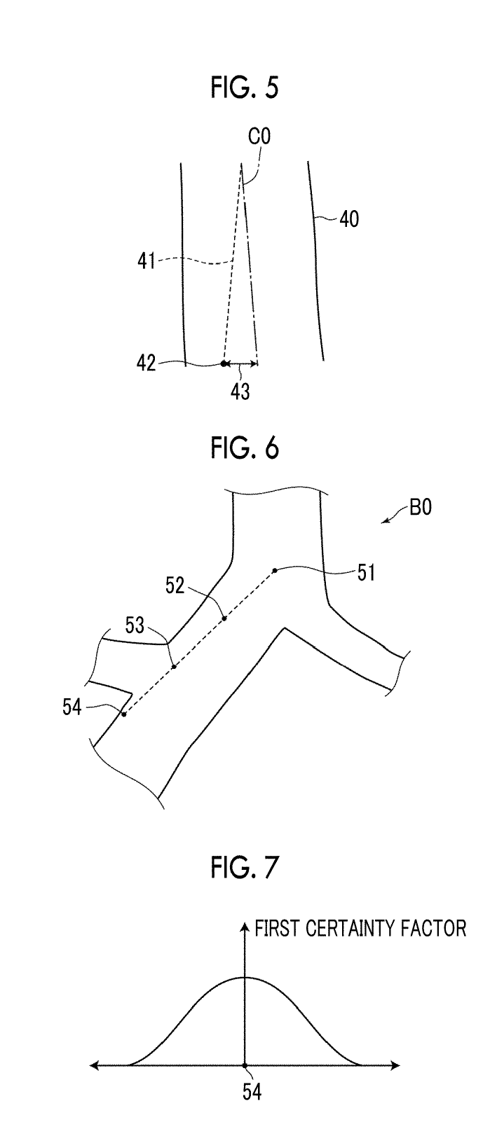

The deviation calculation section 35 calculates the deviation of the endoscope distal end 3B within the bronchus based on the amount of movement stored in the storage 13. FIG. 5 is a diagram illustrating the calculation of the deviation of the endoscope distal end 3B. A bronchus 40 and its central axis C0 are shown in FIG. 5. It is preferable that the endoscope distal end 3B moves through the center axis C0 of the bronchus 40. In practice, however, the endoscope distal end 3B moves with a distance from the central axis C0 as indicated by a broken line 41. In the present embodiment, based on the amount of parallel movement among the amounts of movement stored in the storage 13, the distance of the endoscope distal end 3B from the central axis C0 is calculated as the deviation of the endoscope distal end 3B within the bronchus. As shown in FIG. 5, in a case where the endoscope distal end 3B is located at a position 42, the deviation is expressed by 43.

The position estimation section 36 estimates the position of the endoscope distal end 3B within the bronchus based on the amount of movement of the endoscope distal end 3B from the acquisition position of the second endoscope image Gt-1 to the acquisition position of the first endoscope image Gt and the deviation of the endoscope distal end 3B calculated by the deviation calculation section 35. FIG. 6 is a diagram illustrating the estimation of the position of the endoscope distal end. In FIG. 6, the initial position of the endoscope distal end 3B in the bronchus image B0 is set as a position 51. The endoscope distal end 3B moves from the initial position 51 toward the back of the bronchus with a deviation with respect to a position 52, a position 53, and a position 54. In a case where the acquisition position of the second endoscope image Gt-1 is the position 53 and the acquisition position of the first endoscope image Gt is the position 54, the position estimation section 36 estimates the position 54 as the position of the endoscope distal end 3B.

The position estimation section 36 calculates the first certainty factor A1 indicating the possibility of presence of the endoscope distal end 3B with the estimated position of the estimated endoscope distal end 3B as a reference. The first certainty factor A1 has a three-dimensional distribution with the estimated position of the endoscope distal end 3B as a reference, and has a larger value as a distance from the estimated position becomes smaller. In the present embodiment, it is assumed that the first certainty factor A1 has a value of 0 to 1. The first certainty factor A1 has been experimentally calculated in advance and stored in the storage 13. As the time from the acquisition of the reference endoscope image to the acquisition of the latest endoscope image becomes longer, the first certainty factor A1 becomes smaller and its distribution also becomes different. Therefore, in the present embodiment, a plurality of types of first certainty factors A1 are stored in the storage 13 according to the time from the acquisition of the reference endoscope image to the acquisition of the latest endoscope image. The position estimation section 36 acquires the first certainty factor A1 corresponding to the time from the acquisition of the reference endoscope image to the acquisition of the latest endoscope image (in the present embodiment, the time from the acquisition of the second endoscope image Gt-1 to the acquisition of the first endoscope image Gt) from the storage 13.

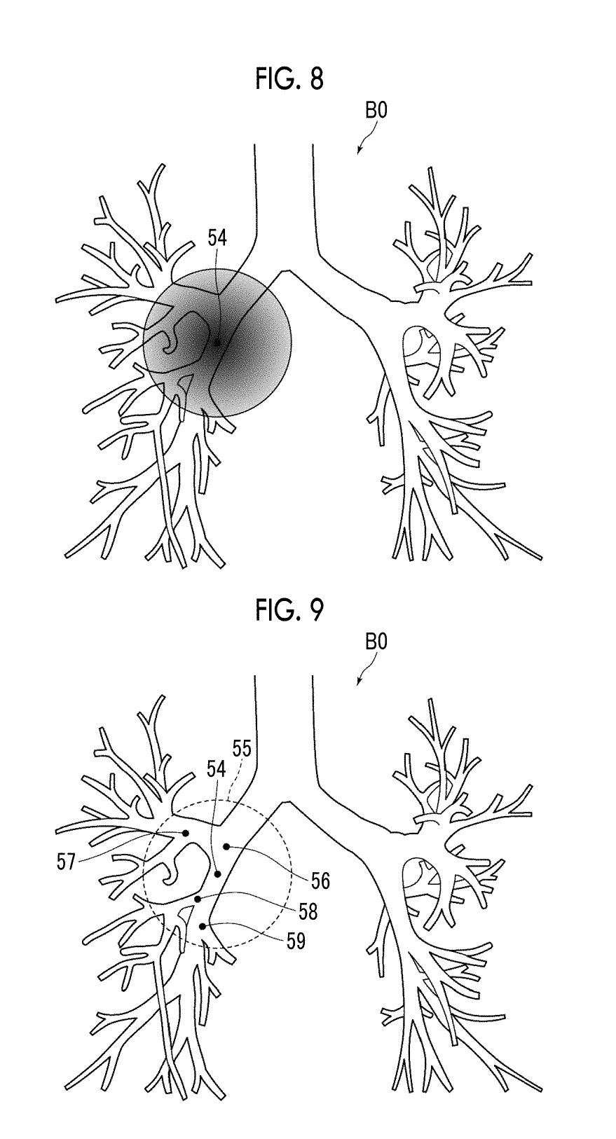

FIG. 7 is a diagram showing the distribution of the first certainty factor A1. In FIG. 7, the horizontal axis indicates a position, and the vertical axis indicating the magnitude of the first certainty factor A1. Although FIG. 7 is shown in two dimensions for the purpose of explanation, the first certainty factor A1 has a three-dimensional distribution. As shown in FIG. 7, the first certainty factor A1 has the highest value at the estimated position 54 of the endoscope distal end 3B, and the value becomes smaller as the distance from the position 54 increases. Therefore, the first certainty factor A1 has a spherical distribution centered on the estimated position 54 in the bronchus image B0 shown in FIG. 8.

The second certainty factor calculation unit 24 calculates a second certainty factor A2, which indicates the possibility of presence of the endoscope distal end 3B, at each of a plurality of positions in the bronchus image B0 by performing matching between the bronchus image B0 and the endoscope image G0 at each of a plurality of positions in the bronchus. Therefore, the second certainty factor calculation unit 24 performs matching between the first endoscope image Gt and the bronchus image B0 first. It is difficult to match the first endoscope image Gt at all pixel positions within the bronchus in the bronchus image B0 from the viewpoint of the amount of calculation and the calculation time. Therefore, in the present embodiment, matching is performed at discrete positions in the bronchus image B0. For example, matching may be performed at a predetermined pixel interval on the central axis C0 in the bronchus image B0, or matching may be performed only within a predetermined pixel range centered on the branching position in the bronchus image B0. Alternatively, matching may be performed only within a predetermined range including the position of the endoscope distal end 3B estimated by the position estimation section 36 or the position of the endoscope distal end 3B specified in the previous processing. Alternatively, matching may be performed by combining these matching methods. In the present embodiment, it is assumed that matching is performed within a predetermined range including the position of the endoscope distal end 3B estimated by the position estimation section 36.

The second certainty factor calculation unit 24 first sets the position of the endoscope distal end 3B, which is estimated by the position estimation section 36 of the first certainty factor calculation unit 23, in the bronchus image B0, and generates a virtual branch image within a predetermined range with the set position as a reference. FIG. 9 is a diagram showing a range for generating a virtual branch image. As shown in FIG. 9, in a case where it is estimated that the endoscope distal end 3B is located at the position 54, the second certainty factor calculation unit 24 generates a virtual branch image in a spherical range 55 centered on the position 54. Specifically, the second certainty factor calculation unit 24 specifies the position of the branch of the bronchus image B0 within the range 55, detects a hole portion of the branch in a direction in which the endoscope distal end 3B is directed from the specified position, and generates a virtual branch image configured to include only the contour of the hole portion. For the sake of explanation, in FIG. 9, it is assumed that a virtual endoscope image is generated at positions 56 to 59 of four branches within the range 55.

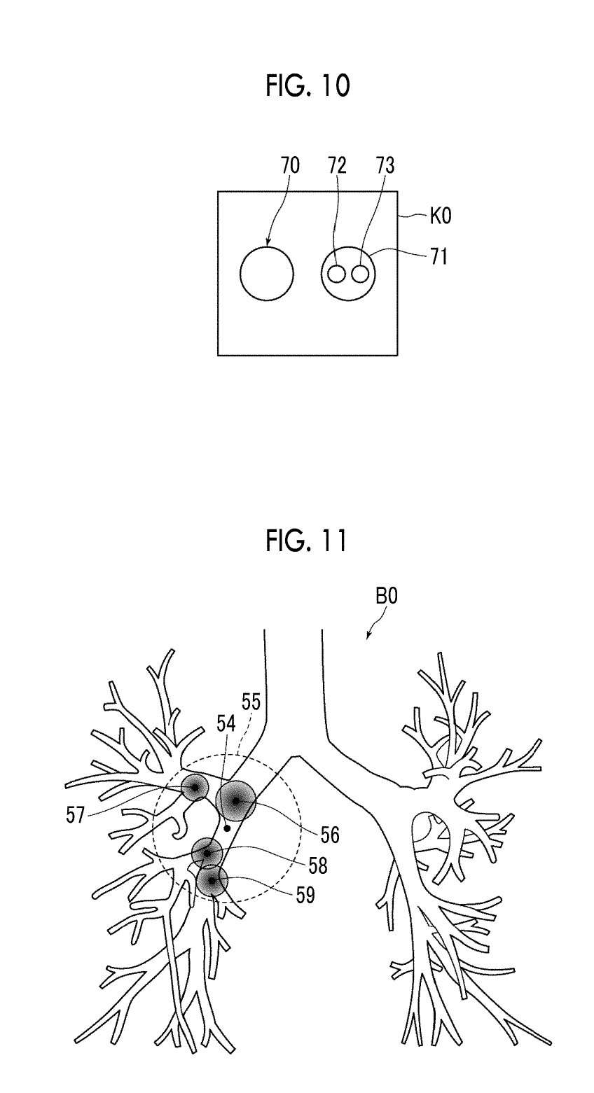

FIG. 10 is a diagram showing a virtual branch image. As shown in FIG. 10, contours 70 to 73 of hole portions of branches are included in a virtual branch image K0. Since a plurality of branches are included in the range 55, a plurality of virtual branch images are generated.

The second certainty factor calculation unit 24 performs matching between the first endoscope image Gt and the virtual branch image K0 by calculating the correlation between the first endoscope image Gt and all the virtual branch images K0. As the correlation, it is possible to use the inverse of the sum of absolute values of differences between pixel values, the inverse of the sum of squares of differences between pixel values, and the like. In the present embodiment, the calculated correlation is the second certainty factor A2. Correlation is also calculated at positions around the positions 56 to 59 of the branches where the virtual branch image K0 is generated. As a result, the second certainty factor A2 has a distribution in which the value is highest at the positions 56 to 59 of the branches where the virtual branch image K0 is generated and the value becomes small as the distances from the positions 56 to 59 increase.

FIG. 11 is a diagram illustrating the second certainty factor. As shown in FIG. 11, in a case where it is estimated that the endoscope distal end 3B is located at the position 54, the second certainty factor calculation unit 24 calculates the second certainty factor A2 at the positions 56 to 59 within the spherical range 55 centered on the position 54 in the bronchus image B0. FIG. 11 shows that the second certainty factor A2 is large for the center of a circle having a high density.

The current position specifying unit 25 specifies the current position of the endoscope distal end 3B based on the first certainty factor A1 and the second certainty factor A2. Specifically, the current position specifying unit 25 specifies adds up the first certainty factor A1 and the second certainty factor A2 in the bronchus image B0, and specifies a pixel position in the bronchus image B0, at which the sum of the first certainty factor A1 and the second certainty factor A2 is the largest, as the current position of the endoscope distal end 3B.

Here, it is assumed that the values of the second certainty factor A2 at the positions 56 to 59 are 0.7, 0.5, 0.4, and 0.2, respectively. In addition, it is assumed that the first certainty factor A1 has a distribution centered on the position 54 and the values of the first certainty factor A1 at the positions 56 to 59 are 0.6, 0.5, 0.8, and 0.5, respectively. The sum of the first certainty factor A1 and the second certainty factor A2 at the positions 56 to 59 is 1.3, 1.0, 1.2, and 0.7, respectively. Therefore, the current position specifying unit 25 specifies the position 56 where the sum is the largest as the current position of the endoscope distal end 3B.

The display control unit 26 connects the current position of the endoscope distal end 3B specified for each endoscope image G0, and displays the result on the bronchus image B0 displayed on the display 14.

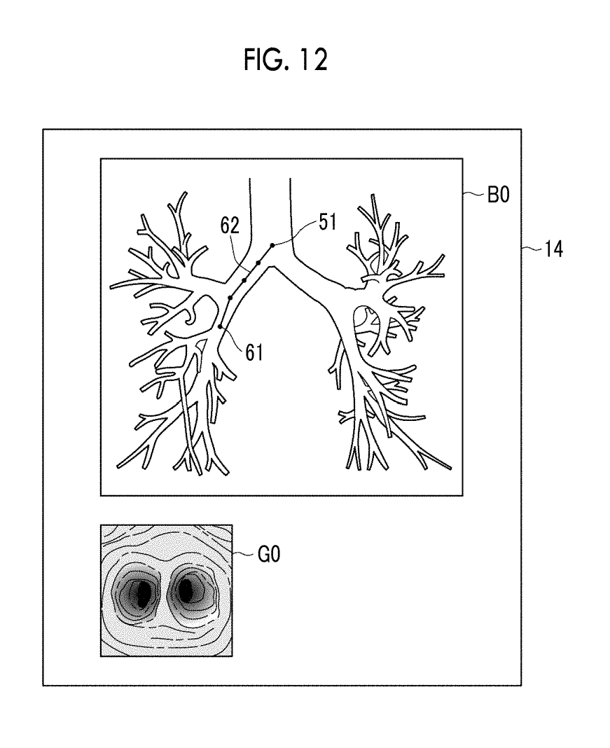

FIG. 12 is a diagram showing a bronchus image displayed on the display. As shown in FIG. 12, the bronchus image B0 and the endoscope image G0 captured at the current position are displayed on the display 14. The endoscope image G0 is the first endoscope image Gt. In the bronchus image B0, an initial position 51 and a current position 61 of the endoscope distal end 3B and a trajectory 62 up to the current position 61, which is obtained by connecting the current position of the endoscope distal end 3B specified between the initial position 51 and the current position 61, are displayed. The distal end of the trajectory 62 is the current position 61 of the endoscope distal end 3B. In addition, for example, the current position 61 of the endoscope distal end 3B may blink or a mark may be given thereto, so that the position of the endoscope distal end 3B can be viewed in the bronchus image B0.

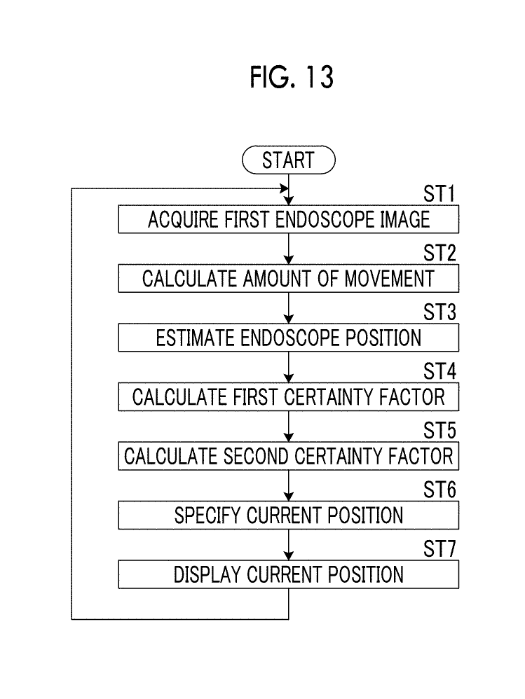

Next, the process performed in the first embodiment will be described. FIG. 13 is a flowchart showing the process performed in the first embodiment. Here, the process in a case where the endoscope distal end 3B is inserted from the initial position toward the back of the bronchus and the endoscope image G0 at a certain point in time is the first endoscope image Gt will be described. In addition, it is assumed that the bronchus image B0 is generated from the three-dimensional image V0 by the bronchus image generation unit 22. The image acquisition unit 21 acquires the endoscope image G0 at a certain point in time as the first endoscope image Gt (step ST1). The first certainty factor calculation unit 23 calculates the amount of movement of the endoscope distal end 3B from the position of the endoscope distal end 3B specified in a case where the second endoscope image Gt-1 is acquired at the immediately preceding time (step ST2). The position of the endoscope is estimated based on the calculated amount of movement (step ST3). The first certainty factor A1 indicating the possibility of presence of the endoscope distal end 3B within the bronchus is calculated based on the estimated position (step ST4).

Then, the second certainty factor calculation unit 24 calculates the second certainty factor A2, which indicates the possibility of presence of the endoscope distal end 3B, at each of a plurality of positions in the bronchus image B0 by performing matching between the bronchus image B0 and the first endoscope image Gt at each of a plurality of positions in the bronchus (step ST5). Then, the current position specifying unit 25 specifies the current position of the endoscope distal end 3B based on the first certainty factor A1 and the second certainty factor A2 (step ST6). Then, the display control unit 26 displays the specified current position of the endoscope distal end 3B on the bronchus image B0 displayed on the display 14 (step ST7), and the process returns to step ST1. The specified current position of the endoscope distal end 3B is stored in the storage 13, and is used as a position where an endoscope image serving as a reference in the next processing is acquired.

Using the first certainty factor A1, a relative change in the position of the endoscope distal end 3B from the previous position can be accurately calculated. However, as the time passes, an error may be accumulated to lower the accuracy. On the other hand, using the second certainty factor A2, the absolute position of the endoscope distal end 3B can be accurately calculated. However, a plurality of branches having similar shapes are included in the bronchus. For this reason, the second certainty factor A2 is large at a plurality of positions within the bronchus. As a result, there is a possibility that the current position of the endoscope distal end 3B cannot be specified.

In the present embodiment, the current position of the endoscope distal end 3B is specified based on both the first certainty factor A1 and the second certainty factor A2. Therefore, by taking advantage of the first certainty factor A1 and the second certainty factor A2, it is possible to more accurately specify the position of the endoscope distal end 3B within the bronchus.

In addition, by calculating the second certainty factor A2 in a predetermined range with the position of the endoscope estimated by the first certainty factor calculation unit 23 as a reference, it is possible to narrow the calculation range of the second certainty factor A2. Therefore, it is possible to quickly calculate the second certainty factor A2 by reducing the amount of calculation.

In the first embodiment described above, the second endoscope image Gt-1 acquired before the first endoscope image Gt, which is the latest endoscope image, is acquired is a reference endoscope image. However, the reference endoscope image is not limited to the second endoscope image Gt-1. For example, an endoscope image acquired at the initial position 51 may be used as the reference endoscope image. In this case, the first certainty factor A1 is calculated based on the endoscope image acquired at the initial position 51 and the latest first endoscope image Gt. In addition, an endoscope image Gt-n n frames (n is a plural number) before the first endoscope image Gt, which is the latest endoscope image, is acquired may be used as the reference endoscope image. In this case, the first certainty factor A1 is calculated based on the first endoscope image Gt and the endoscope image Gt-n n frames before the first endoscope image Gt.

In the first embodiment described above, the first certainty factor A1 is calculated based on the first endoscope image Gt and the second endoscope image Gt-1. However, a plurality of reference endoscope images may be set, and a plurality of first certainty factors A1 may be calculated based on each of the plurality of reference endoscope images and the latest first endoscope image Gt. Hereinafter, this will be described as a second embodiment. An endoscope position specifying device according to the second embodiment has the same configuration as the endoscope position specifying device according to the first embodiment, and only the processing to be performed is different. Accordingly, the detailed explanation of the device will be omitted herein.

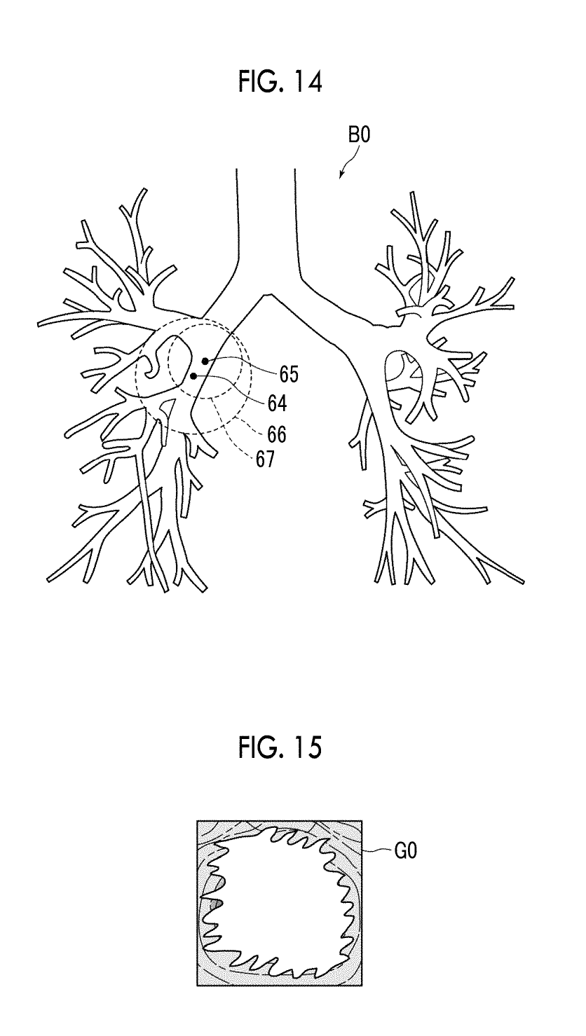

FIG. 14 is a diagram showing the position of the endoscope estimated based on a plurality of reference endoscope image in the second embodiment. Here, it is assumed that two endoscope positions are estimated based on two reference endoscope images. For example, it is assumed that one of the reference endoscope images is the second endoscope image Gt-1 similar to the above embodiment and the other one is an endoscope image Gt-10 10 frames before the first endoscope image Gt.

The first certainty factor calculation unit 23 estimates the position of the endoscope distal end 3B based on the first endoscope image Gt and the second endoscope image Gt-1. This is assumed to be a first position 64 of the endoscope distal end 3B. The first certainty factor calculation unit 23 estimates the position of the endoscope distal end 3B based on the first endoscope image Gt and the endoscope image Gt-10. This is assumed to be a second position 65 of the endoscope distal end 3B. In this case, at each of the first and second positions 64 and 65, first certainty factors A1-1 and A1-2 having a distribution are calculated. The first certainty factor decreases as the time interval between the two endoscope images for estimating the position of the endoscope distal end 3B increases. Therefore, as shown in FIG. 14, the distribution range 66 of the first certainty factor A1-1 is larger than the distribution range 67 of the first certainty factor A1-2. Although not shown, the value of the first certainty factor A1-1 is larger than the value of the first certainty factor A1-2.

In this case, the current position specifying unit 25 estimates the current position of the endoscope distal end 3B based on the first certainty factor A1-1, the first certainty factor A1-2, and the second certainty factor A2. Here, it is assumed that the values of the second certainty factor A2 at the positions 56 to 59 shown in FIG. 9 are 0.7, 0.5, 0.4, and 0.2, respectively, as in the first embodiment. In addition, it is assumed that the first certainty factor A1-1 has a distribution centered on the position 64 and the values of the first certainty factor A1-1 at the positions 56 to 59 are 0.6, 0.5, 0.8, and 0.5, respectively. In addition, it is assumed that the values of the first certainty factor A1-2 at the positions 56 to 59 are 0.4, 0.4, 0.3, and 0.6, respectively. The sum of the first certainty factor A1-1 and the second certainty factor A2 at the positions 56 to 59 is 1.3, 1.0, 1.2, and 0.7, respectively. The sum of the first certainty factor A1-2 and the second certainty factor A2 at the positions 56 to 59 is 1.1, 0.9, 0.7, and 0.8, respectively. Therefore, the current position specifying unit 25 specifies the position 56 where the sum is the largest as the current position of the endoscope distal end 3B.

Also in the second embodiment, in a case where the reference endoscope image is temporally close to the first endoscope image Gt, the first certainty factor is high. On the other hand, in an endoscopic examination, there is a case where the inner wall of the bronchus is imaged by bending the endoscope distal end 3B. In such a case, the endoscope image G0 does not include a hole portion. For this reason, the first certainty factor calculation unit 23 cannot detect a hole portion from the endoscope image G0. As a result, it is not possible to calculate the first certainty factor. The first certainty factor calculation unit 23 can calculate the first certainty factor by estimating the amount of movement of the endoscope distal end 3B and the position of the endoscope distal end 3B by performing matching between the first endoscope image Gt and the second endoscope image Gt-1 without detecting a hole portion. In this case, the accuracy is lower than that in a case where a hole portion is used.

In an endoscopic examination, there is a case where drug is sprayed from the endoscope distal end 3B for treatment or the like. In an endoscope image obtained during the spraying of drug, no hole portion is viewed as shown in FIG. 15. Accordingly, the endoscope image obtained during the spraying of drug is an abnormal endoscope image that is meaningless from the medical point of view. Even if such an abnormal endoscope image is used as a reference endoscope image, the position of the endoscope distal end 3B cannot be accurately estimated. As a result, the accuracy of the first certainty factor A1 is also low.

As in the second embodiment, by setting a plurality of reference endoscope images and calculating a plurality of first certainty factors A1 based on each of the plurality of reference endoscope images and the latest first endoscope image Gt, it is possible to reduce a possibility that a reference endoscope image will become an abnormal endoscope image or an image not including a hole portion. For this reason, by estimating the position of the endoscope more accurately, it is possible to calculate the first certainty factor with higher accuracy. Therefore, the current position of the endoscope distal end 3B can be specified more accurately.

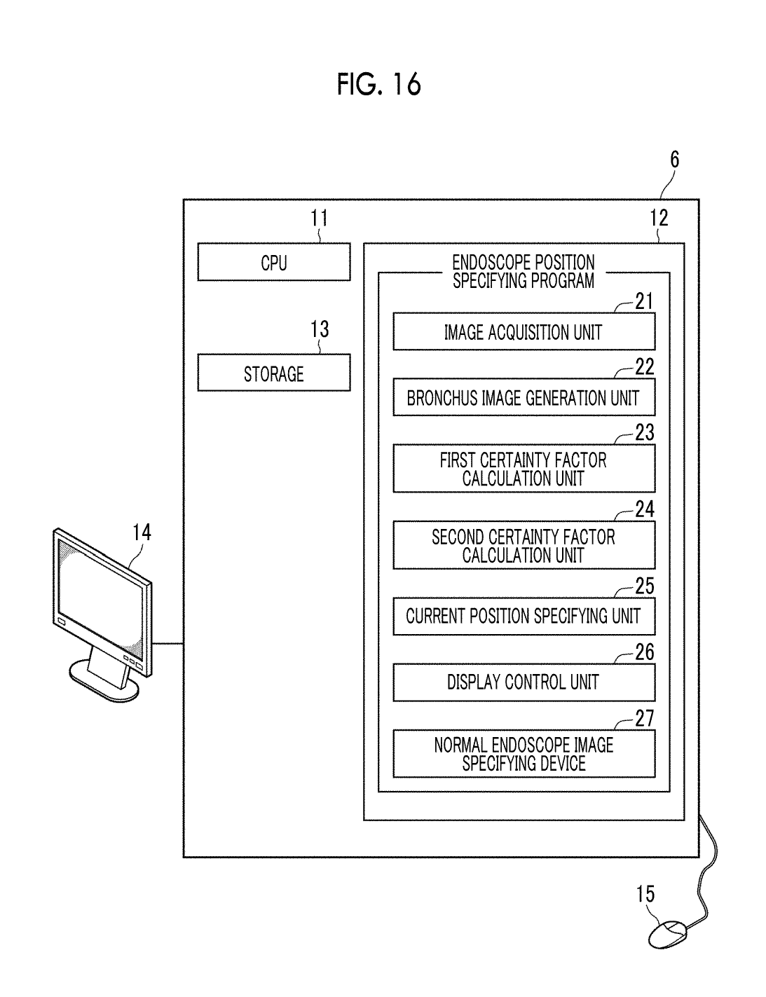

Next, a third embodiment of the invention will be described. FIG. 16 is a diagram showing the schematic configuration of an endoscope position specifying device according to the third embodiment. In FIG. 16, the same components as in FIG. 2 are denoted by the same reference numbers, and the detailed explanation thereof will be omitted. The endoscope position specifying device according to the third embodiment is different from the endoscope position specifying device according to the first embodiment in that a normal endoscope image specifying device 27 for specifying normal endoscope images among sequentially acquired endoscope images is further provided and the first certainty factor calculation unit 23 calculates the first certainty factor A1 by selecting a reference endoscope image and the latest endoscope image from the normal endoscope images.

The normal endoscope image specifying device 27 determines whether or not a hole portion is included in each of the sequentially acquired endoscope images. The normal endoscope image specifying device 27 specifies an endoscope image, which is determined to include a hole portion, as a normal endoscope image. Alternatively, the normal endoscope image specifying device 27 may determine whether or not a hole portion is included for all of the sequentially acquired endoscope images, or may determine whether or not a hole portion is included by appropriately thinning out the endoscope images.

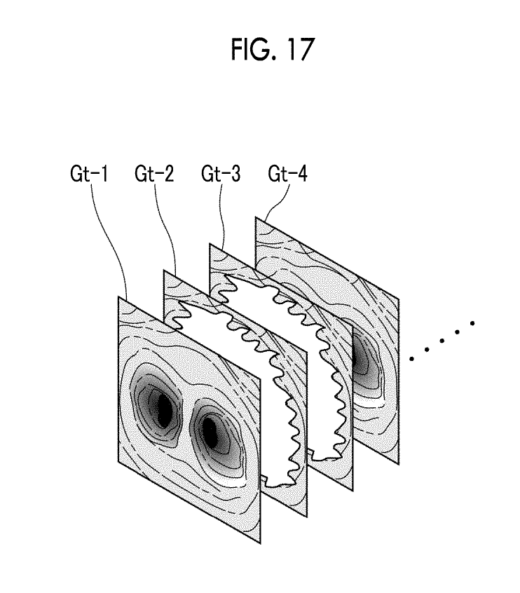

FIG. 17 is a diagram illustrating how to specify a normal endoscope image. As shown in FIG. 17, it is assumed that endoscope images Gt-2 and Gt-3, among the sequentially acquired endoscope images Gt-4, Gt-3, Gt-2, and Gt-1, are abnormal endoscope images. The normal endoscope image specifying device 27 determines whether or not a hole portion is included in each of the endoscope images Gt-4, Gt-3, Gt-2, and Gt-1. In this case, the endoscope images Gt-2 and Gt-3 are abnormal endoscope images not including a hole portion. Therefore, the normal endoscope image specifying device 27 specifies the endoscope images Gt-1 and Gt-4 as normal endoscope images. In this case, the first certainty factor calculation unit 23 selects the endoscope image Gt-1 as the latest endoscope image, and selects the endoscope image Gt-4 as a reference endoscope image. Then, the first certainty factor calculation unit 23 calculates the first certainty factor A1 based on the endoscope images Gt-1 and Gt-4.

As described above, by calculating the first certainty factor by specifying normal endoscope images among the sequentially acquired endoscope images and selecting the reference endoscope image and the latest endoscope image from the normal endoscope images, it is possible to accurately estimate the position of the endoscope without being affected by the abnormal endoscope images.

In the third embodiment described above, a normal endoscope image is specified by determining whether or not a hole portion is detected in the endoscope image. However, a normal endoscope image may be specified from sequentially acquired endoscope images using a discriminator learned to discriminate between a normal endoscope image and an abnormal endoscope image.

In each embodiment described above, the hole portion detection section 31 of the first certainty factor calculation unit 23 detects a hole portion from each of the first and second endoscope images. However, a hole portion may also be detected from one of the first and second endoscope images Gt and Gt-1. For example, in a case where a hole portion is detected only from the first endoscope image Gt, an image in which the detected hole portion is cut out or an image in which the weight of the hole portion is increased can be generated, and the first parameter P1 and the second parameter P2 can be calculated by using such an image and the second endoscope image Gt-1.

In each embodiment described above, the first certainty factor calculation unit 23 estimates the position of the endoscope distal end 3B by detecting a hole portion from the first and second endoscope images Gt and Gt-1. However, the position of the endoscope distal end 3B may also be estimated by performing matching between the first endoscope image Gt and the second endoscope image Gt-1 without detecting a hole portion. Thus, even in a case where the endoscope distal end 3B is bent to image the inner wall of the bronchus, the position of the endoscope distal end 3B can be estimated although the accuracy is low. In a case where the first endoscope image Gt or the second endoscope image Gt-1 is an abnormal endoscope image, it is not possible to calculate the second certainty factor A2. In this case, although the accuracy is low, the position of the endoscope distal end 3B estimated without detecting a hole portion by the first certainty factor calculation unit 23 can be set as the current position of the endoscope distal end 3B.

In each embodiment described above, the amount of movement is accumulated and stored in the storage 13 every time the endoscope image G0 is acquired from the initial position by the first certainty factor calculation unit 23. Here, the amount of movement is accumulated and stored in order to determine in which direction the endoscope distal end 3B is directed at the branch of the bronchus. Therefore, the accumulated amount of movement may be reset to 0 every time the endoscope distal end 3B passes the branch, and the amount of movement may be accumulated and stored only from the passed branch to the next branch to calculate the first certainty factor A1.

In each embodiment described above, the second parameter P2 includes the amount of rotation. However, the second parameter P2 including only the amount of enlargement and reduction may be calculated.

In each embodiment described above, the deviation of the endoscope is calculated based on the stored amount of movement, and the position of the endoscope is displayed based on the amount of movement and the deviation. However, the position of the endoscope may be displayed based only on the amount of movement without calculating the deviation of the endoscope.

In each embodiment described above, the case has been described in which the endoscope position specifying device of the invention is applied to the observation of the bronchus. However, without being limited thereto, the invention can also be applied to a case of observing a tubular structure having branch structures, such as blood vessels, with an endoscope.

Hereinafter, the effect of the embodiment of the invention will be described.

By calculating the second certainty factor in a predetermined range with the estimated position of the endoscope as a reference, it is possible to narrow the calculation range of the second certainty factor. Therefore, it is possible to quickly calculate the second certainty factor by reducing the amount of calculation for calculating the second certainty factor.

By calculating the first certainty factor by specifying normal endoscope images among the sequentially acquired endoscope images and selecting the reference endoscope image and the latest endoscope image from the normal endoscope images, it is possible to accurately estimate the position of the endoscope without being affected by the abnormal endoscope images.

By setting a plurality of reference endoscope images, estimating a plurality of amounts of movement of the endoscope during a period from the acquisition of each of the plurality of reference endoscope images to the acquisition of the latest endoscope image, estimating a plurality of endoscope positions from the plurality of amounts of movement, and calculating the first certainty factor at each of the plurality of estimated positions, it is possible to estimate the position of the endoscope more accurately.

What is claimed is:

1. An endoscope position specifying device, comprising:
endoscope image acquisition unit for sequentially acquiring endoscope images that are generated by an endoscope inserted into a tubular structure having a plurality of branch structures and that show an inner wall of the tubular structure;
image generation unit for generating an image of the tubular structure from a three-dimensional image including the tubular structure;
first certainty factor calculation unit for calculating an amount of movement of the endoscope during a period from acquisition of a reference endoscope image to acquisition of a latest endoscope image based on the sequentially acquired endoscope images, estimating a position of the endoscope based on the calculated amount of movement, and calculating a first certainty factor indicating a possibility of presence of the endoscope within the tubular structure based on the estimated position;
second certainty factor calculation unit for calculating a second certainty factor, which indicates a possibility of presence of the endoscope, at each of a plurality of positions within the tubular structure by performing matching between the image of the tubular structure and each of the endoscope images at each of the plurality of positions within the tubular structure; and
current position specifying unit for specifying a current position of the endoscope based on the first and second certainty factors.

2. The endoscope position specifying device according to claim 1,
wherein the second certainty factor calculation unit calculates the second certainty factor in a predetermined range with the position of the endoscope estimated by the first certainty factor calculation unit as a reference.

3. The endoscope position specifying device according to claim 1, further comprising:
normal endoscope image specifying unit for specifying normal endoscope images among the sequentially acquired endoscope images,
wherein the first certainty factor calculation unit calculates the first certainty factor by selecting the reference endoscope image and the latest endoscope image from the normal endoscope images.

4. The endoscope position specifying device according to claim 1,
wherein the first certainty factor calculation unit sets a plurality of the reference endoscope images, calculates a plurality of amounts of movement of the endoscope during a period from acquisition of each of the plurality of reference endoscope images to acquisition of the latest endoscope image, estimates a plurality of positions of the endoscope from the plurality of amounts of movement, and calculates the first certainty factor at each of the plurality of estimated positions, and
the current position specifying unit specifies the current position of the endoscope based on a plurality of the first certainty factors and the second certainty factors.

5. The endoscope position specifying device according to claim 1, further comprising:
display control unit for displaying the image of the tubular structure and displaying the current position of the endoscope on the image of the tubular structure.

6. An endoscope position specifying method, comprising:
sequentially acquiring endoscope images that are generated by an endoscope inserted into a tubular structure having a plurality of branch structures and that show an inner wall of the tubular structure;
generating an image of the tubular structure from a three-dimensional image including the tubular structure;
calculating an amount of movement of the endoscope during a period from acquisition of a reference endoscope image to acquisition of a latest endoscope image based on the sequentially acquired endoscope images, estimating a position of the endoscope based on the calculated amount of movement, and calculating a first certainty factor indicating a possibility of presence of the endoscope within the tubular structure based on the estimated position;
calculating a second certainty factor, which indicates a possibility of presence of the endoscope, at each of a plurality of positions within the tubular structure by performing matching between the image of the tubular structure and each of the endoscope images at each of the plurality of positions within the tubular structure; and
specifying a current position of the endoscope based on the first and second certainty factors.

7. A non-transitory computer-readable recording medium having stored therein an endoscope position specifying program causing a computer to execute:
a step of sequentially acquiring endoscope images that are generated by an endoscope inserted into a tubular structure having a plurality of branch structures and that show an inner wall of the tubular structure;
a step of generating an image of the tubular structure from a three-dimensional image including the tubular structure;
a step of calculating an amount of movement of the endoscope during a period from acquisition of a reference endoscope image to acquisition of a latest endoscope image based on the sequentially acquired endoscope images, estimating a position of the endoscope based on the calculated amount of movement, and calculating a first certainty factor indicating a possibility of presence of the endoscope within the tubular structure based on the estimated position;
a step of calculating a second certainty factor, which indicates a possibility of presence of the endoscope, at each of a plurality of positions within the tubular structure by performing matching between the image of the tubular structure and each of the endoscope images at each of the plurality of positions within the tubular structure; and
a step of specifying a current position of the endoscope based on the first and second certainty factors.
